(19) United States
(12) Patent Application Publication
(10) Pub. No.: US 2011/0319792 A1
(43) Pub. Date: Dec. 29, 2011

(54) APPARATUS AND METHOD FOR MEASURING IN VIVO BIOMECHANICAL PROPERTIES OF SKIN

(75) Inventors: Keng Hui Lim, Singapore (SG); Timothy Poston, Bangalore (IN); Hoan Nghia Ho, Singapore (SG); Chee Meng Chew, Singapore (SG); Chao Yu Peter Chen, Singapore (SG); Sujeevini Jeyapalina, Singapore (SG); Beng Hai Lim, Singapore (SG)

(73) Assignee: National University of Singapore

(21) Appl. No.: 12/964,535

(22) Filed: Dec. 9, 2010

Related U.S. Application Data
(62) Division of application No. 11/993,981, filed on Dec. 27, 2007.
(60) Provisional application No. 60/695,747, filed on Jun. 30, 2005.

(51) Int. Cl. A61B 5/11

(52) U.S. Cl. 600/595

(57) ABSTRACT

An assembly for measuring in vivo biomechanical properties of skin, comprising a testing device, said testing device comprising; a first pad attachable to the skin a second pad attachable to the skin, at a known distance from the first pad; said attachability of the pads to the skin to prevent relative movement between the respective pad and the skin to which it is attached; a forcing means for applying a force to the first pad, whilst said pads are attached to the skin, along a first axis connecting the first and second pad, to induce a corresponding relative movement between the pads due to deformation of the skin between said pads; a force measurement device for measuring the applied force, and; a displacement measurement device for measuring the corresponding induced movement.

### CROSS REFERENCE TO RELATED APPLICATIONS

This application is a divisional application of U.S. application Ser. No. 11/993,981, filed Dec. 27, 2007, which is itself a 35 U.S.C. 371 national phase application of International Application No. PCT/SG2006/000182, filed Jun. 29, 2006, which International Application was published by the International Bureau in English on Jan. 11, 2007, and claims priority to U.S. Application No. 60/695,747, filed Jun. 30, 2005, all of which are incorporated herein by reference in their entirety.

### FIELD OF INVENTION

The invention relates to measurement of biomechanical properties of skin using a noninvasive approach.

### BACKGROUND

Human skin provides the body with a flexible barrier to the exterior environment through a highly integrated layered structure consisting of epidermis, dermis and subcutaneous tissues. Each layer has its own specific structure and functions. Mechanical behaviour of the human skin is complex and well known to exhibit nonlinear and time-dependent mechanical behaviour.

During skin flap/graft reconstruction surgery, surgeons need to transplant a skin graft from a healthy area (i.e., the donor site) to the trauma area (i.e., the recipient site). For a graft, surgeons need to estimate the final shape of an excised flap from the donor site so that it can fit the recipient site. When excised from a donor site, a flap will shrink. The amount of shrinkage is highly sensitive to the patient-specific skin structure.

As widely accepted, skin is biaxially stretched in one's body and thus, one way of estimating the shrinkage is to determine the un-stretched length/natural length (NL) of the skin at various directions. At this stretched state, skin would have residual tension; static and dynamic. The static tension is the built-in skin tension and the dynamic tension is caused by forces from joint movements and other voluntary muscle activity. Both are shown to contribute to skin flap shrinkage. Therefore, in order to predict the patient specific skin flap shrinkage, one would have to measure not only the biomechanical properties but also the natural tension (NT) of a skin site of interest. Some researchers have estimated the natural tension of a skin using a pre-tension apparatus and a strain gauge and reported that the tension is greater in the Langer's line direction. However, at present, there is no commercial device available that will estimate these directionally dependent NT and NL values.

The usual graft is a ‘flap’, a technical term including not only skin but material from beneath it; including blood vessels that microsurgery can connect to vessels at the recipient site. In the present submission, we refer for brevity to this complex multilayer as ‘skin’. From the standpoint of those wishing to measure the mechanical properties of skin in the narrower sense (for example, in assessing the influence on it of a skin cream), the in vivo mechanical effect of the underlying layers is a problem. From a standpoint concerned with grafts, a collective characterisation approximating the combined biomechanics of the multiple layers in a flap is more useful.

A skin flap has two main layers (dermis and fat) with an artery and a returning vein to provide nutrients and remove waste respectively. For survival after grafting, the blood pressure inside the tissue should be kept above a critical value (32 mm Hg). If the pressure falls below this, blood supply will not be adequate and the transplanted flap will not survive. Re-stretching the flap to the original size compresses its incomplete arterial connections to a point where this fails, so the surgeon has a complex problem of determining the excess amount of flap in various directions to be harvested for a given recipient site, while avoiding wastage.

At present, shrinkage estimation is based on the doctor's skill and experience. A doctor will usually furnish an estimate based on a tactile pinch on the patient's skin to estimate the tension and elasticity, on the patient's physiology, on evaluation of the donor site, and on other factors. For junior surgeons, flap/wound mismatch problems are frequent due to judgment error, lack of quantitative tools, and inadequate understanding of the mechanical behaviour of the skin. Such problems often lead to further complications and trauma to the patient. Therefore, in order to assist the surgeons during the critical stage of skin flap planning, there is a need to develop an appropriate measurement device.

It is known that in the normal physiological state skin is strained. This influences its biomechanical behaviour considerably. The influence of mechanical forces on skin has been examined since 1861, when Langer first reported the existence of lines of tension in skin, this work later repeated by Cox. Cox's lines of tension did not match those of Langer, but both reported the symmetrical nature of these lines of tension in the biomechanical behaviour of human skin. These lines can only be defined by microscopic techniques. In a section cut parallel with these lines, most of the collagenous bundles of the reticular layer are cut longitudinally, while in a section cut across the lines, the bundles are in cross section. A line following the preferred orientation of fibres within the dermal tissue is referred to as a Langer's line in honour of Langer, whose pioneering work led to their discovery.

These tension lines are of interest to the surgeon because an incision made parallel to them heals with a finer scar. An incision across them may set up irregular tensions that result in more noticeable scarring. Furthermore, the shrinkage of excised flap shows a high dependency on these lines of tension. Unfortunately, the directions of Langer's lines are not constant between patients but show significant variations, and may not remain constant at an anatomical site for a specific subject. Langer's lines correspond closely with the crease lines on the surface of the skin in most parts of the body. The precise orientation of fibres defining such lines can only be found by penetrative techniques. Because of their invasive nature, such techniques are not widely applicable.

### STATEMENT OF INVENTION

It is, therefore, an object of the present invention to provide a non-invasive testing method for the measurement of biomechanical properties, which in turn may be used to characterise the Langer's lines and to predict skin flap shrinkage pre-operatively. In a first aspect, the invention provides an assembly for measuring in vivo biomechanical properties of skin, comprising a testing device, said testing device comprising; a first pad attachable to the skin; a second pad attachable to the skin, at a known distance from the first pad; said attachability of the pads to the skin to prevent relative movement between the respective pad and the skin to which it is attached; a forcing means for applying a force to the first pad, whilst said pads are attached to the skin, along a first axis connecting the first and second pad, to induce a corresponding relative movement between the pads due to deformation of the skin between said pads; a force measurement device for measuring the applied force, and; a displacement measurement device for measuring the corresponding induced movement.

In a second aspect, the invention provides an assembly for measuring in vivo biomechanical properties of skin, comprising a testing device, said testing device comprising; a first pad attachable to the skin; a second pad attachable to the skin, at a known distance from the first pad; said attachability of the pads to the skin to prevent relative movement between the respective pad and the skin to which it is attached; a forcing means for applying a force to the first pad, whilst said pads are attached to the skin, along a first axis orthogonal to a second axis connecting the first and second pad, to induce a corresponding relative movement between the pads due to deformation of the skin between said pads; a force measurement device for measuring the applied force, and; a displacement measurement device for measuring the corresponding induced movement.

The present invention may avoid the invasive approach of surgery, in order to obtain the mechanical properties of the skin, by taking an alternative non-invasive approach, through mere attachment of the measurement device to the skin. Whilst a surgical approach may provide additional information, it is unnecessary for the measurement problem solved by the present invention.

It will be appreciated by the skilled addressee that the prevention of relative movement between the skin and the pad is applicable within the effective range of applied force and strain for which the device is intended.

Further, the invention may also provide a more rapid means of surveying a large area of the patient, and so provide a more complete map through repeated measurements at several locations. This may not be practical through a surgical approach, since surgery at one point modifies strain and tensions at locations near it.

This invention will also provide a tool for surgeons who want to predict the skin flap shrinkage pre-operatively. As such, the design of the donor flap to be harvested to optimize the healing process and to reduce the tension related scars can be carried out away from the operative room.

In a preferred embodiment, the testing device may also include a support bracket having the first pad slidingly mounted to the support bracket, and the second pad fixedly mounted to the support bracket; such that the first pad is slidingly movable parallel to the first axis.

In a more preferred embodiment the testing device may also include a third pad attached to the skin and fixedly mounted to the support bracket along the first axis, so as to place the first pad intermediate between the second and third pad. The purpose of the third pad is to insulate the measured skin between the first and second pads from external disturbances. Thus, direct axial force may be applied, and a direct force/elongation characteristic determined more accurately. Additional pads mounted to the support bracket may be used as desired to provide further stability during measurement.

In an alternative embodiment, the testing device may use a second pad attached to the skin and fixedly mounted to the support bracket, such that the second pad is spaced from the first pad along a second axis orthogonal to the first axis. By a similar application of force, the position of the second pad, initially level with the first pad may permit measurement of the shear force/elongation characteristic of the skin.

In either embodiment, the testing device may be a unitary device having the second and third pads fixed to the support bracket and the first pad slidable to a desired position, or when attached to the skin, be slidable to permit localised compression/extension of the skin in order to take appropriate measurements.

This unitary structure may further permit easier reattachment for facilitating multiple readings at multiple locations on the patient. The support bracket may also provide a degree of stability to the testing device during testing. The application of force may be offset from the skin and so will apply a moment about the pads. The use of the support bracket may resist this moment through a high tolerance engagement with the pads, whereby rotational displacement is not permitted. Thus, in this embodiment, any error in rotation or moment may be minimised or avoided.

In a further preferred embodiment, the forcing means may include a constant strain rate actuator for selectively applying the force at a pre-determined strain rate to the skin. The visco-elastic properties of the skin may make it susceptible to an erroneous measurement through a non-uniform application of strain. Further, to standardize measurement, it may be necessary to apply strain at a constant rate, for example, at 0.35 mm/sec. The said actuator may further apply the force through a worm gear, or other suitable high tolerance device to ensure accurate movement of the force applicator.

In a more preferred embodiment, the control of the constant strain rate actuator may be subject to a control system, automatically controlling the application of force, and simultaneously recording the force and displacement. This information may also be instantaneously transcribed to a plotter, stored electronically to a file or both.

In a further preferred embodiment, the pads may be attached to the skin using skin attachment means, said skin attachment means may include any one or a combination of adhesive material, such as double-sided tape or liquid adhesive, clamps to clamp each pad to the skin and a strap for strapping each pad to the skin, attaching it by virtue of the tension in the strap. For instance, the strap may be closed through Velcro™. It may further include a spacer placed beneath the pad between the strap and skin for concentrating a skin attachment force at the pad.

In a more preferred embodiment, the force may be measured by a load cell. This load cell may further be located adjacent the skin in contact with the pad, and preferably in contact with the skin attachment means.

An application of this testing device may include the determination of biomechanical properties of the skin of a patient which may include any one or a combination of linear and shear force-elongation characteristics, and time-dependent force and elongation characteristics, such as force relaxation and creep.

By taking a plurality of measurements of applied force and corresponding induced movement at a plurality of locations, two-dimensional biomechanical properties may be determined, which may include determining the direction of the Langer's Line, biomechanical properties to determine skin flap shrinkage, natural tension and natural length measurements.

In a preferred embodiment the fixed mounting of the second and third pad to the support bracket may be selectively adjustable to permit sliding movement of said pads.

It should be noted that the sources of error may include the inconsistent pressure with which testing device may press onto the skin at the pads, and the handling means used by the operator. Therefore, in a preferred embodiment, the assembly may also include a positioning assembly having an engagement portion for engaging an external body and a holding portion for holding the testing device, said positioning assembly adapted to apply a constant and consistent pressure of the pads on the skin at a specified force.

In a preferred embodiment the holding portion may have a selective sliding engagement with the testing device. Also, the positioning assembly may be selectively deformable for positioning the testing device relative to the skin.

In a more preferred embodiment the holding portion may include a load measurement device to measure the component of force applied at right angles to the skin by the testing device. The load measurement device may also measure the applied torque in order to make sure the pads apply even pressure onto the skin.

In a third aspect, the invention provides a method for measuring in vivo biomechanical properties of skin, comprising the steps of attaching a first pad to the skin; attaching a second pad to the skin, at a known distance from the first pad, said pads attached to prevent relative movement between the respective pad and the skin; applying a force to the first pad, along a first axis connecting the first and second pad, to induce corresponding relative movement between the pads due to deformation of the skin between said pads; measuring the applied force, and; measuring the corresponding induced movement.

In a preferred embodiment the method may include measuring the applied force and the corresponding induced movement in a plurality of directions for the same region of skin, and determining two dimensional biomechanical properties based on measurements in the plurality of directions. In a most preferred embodiment, this may provide sufficient information to determine the direction of the Langer's Line in the said region of skin and other necessary biomechanical properties and natural tension measurements to estimate skin flap shrinkage.

In a fourth aspect, the invention provides a method for measuring in vivo natural length of skin, comprising the steps of: attaching a first pad-to the skin, attaching a second pad to the skin, at a known distance from the first pad, attaching a third pad to the skin, co-linear with a first axis connecting the first and second pad, so as to place the first pad intermediate the second and third pad; said pads attached to prevent relative movement between the respective pad and the skin; applying a force to the first pad, along the first axis towards the third pad, to induce relative movement between the pads to cause a desired deformation of the skin between said pads, up to a pre-determined physical limit, and measuring the applied force on reaching said limit; releasing said force; re-attaching either or both said second and third pads at a pre-determined distance closer to the first pad; re-applying a force to the first pad, along the first axis towards the third pad, to induce relative movement between the pads to a cause a desired deformation of the skin between said pads, up to the pre-determined limit, and measuring the applied force on reaching said limit; releasing said force; repeating a cycle of re-attaching, reapplying, measuring and releasing until a specified criteria for the measured forces is met, the natural length being equal to the distance between the second and third pads when the specified criteria is met.

In a fifth aspect, the invention provides a method for measuring in vivo natural tension of skin, comprising the steps of attaching a first pad to the skin, attaching a second pad to the skin, at a known distance from the first pad, said pads attached to prevent relative movement between the respective pad and the skin; applying a force to the first pad, toward the second pad along a first axis connecting the first and second pad, to induce corresponding relative movement between the pads to cause deformation of the skin between said pads, until the distance between the first and second pads is equal to a natural length of the skin; measuring the applied force, the applied force being equal to the natural tension.

### BRIEF DESCRIPTION OF THE DRAWINGS

It will be convenient to further describe the present invention with respect to the accompanying drawings which illustrate possible arrangements of the invention. Other arrangements of the invention are possible, and consequently the particularity of the accompanying drawings is not to be understood as superseding the generality of the preceding description of the invention.

FIG. 1 is a graphical representation used for locating the Langer's Line;

FIG. 2 is a representation of one approach used for identifying the ellipse of FIG. 1;

FIG. 3 is an isometric view of one embodiment according to the present invention;

FIGS. 4(a) and (b) are views of a second embodiment according to the present invention;

FIG. 5 is an isometric view of a third embodiment according to the present invention;

FIG. 6 is an isometric view of a fourth embodiment according to the present invention;

FIG. 7 is an isometric view of a fifth embodiment according to the present invention;

FIGS. 8(a) and (b) are schematic views of the load distribution of the skin according to the present invention;

FIGS. 9(a) and (b) are plan views of a sixth embodiment of the present invention;

FIGS. 10(a) to (d) are sequential views of a method according to a further embodiment of the present invention;

FIGS. 11(a) to (d) are sequential views of a method according to a further embodiment of the present invention;

FIGS. 12(a) and (b) are experimental results from conducting the methods of FIGS. 10 and 11, and;

FIGS. 13(a) and (b) are sequential views of a method according to a further embodiment of the present invention.

### DESCRIPTION OF PREFERRED EMBODIMENT

It has been reported that the load in the high modulus region is primarily due to the stretching of collagen fibres, drawn tight, whereas deformation of the elastin network governs behaviour in the low modulus region/initial phase, where a typical collagen molecule is sufficiently slack to represent little resistance to skin stretching. Therefore, by studying the high modulus region of the force-elongation curve, it is possible to attain information on the collagen structure.

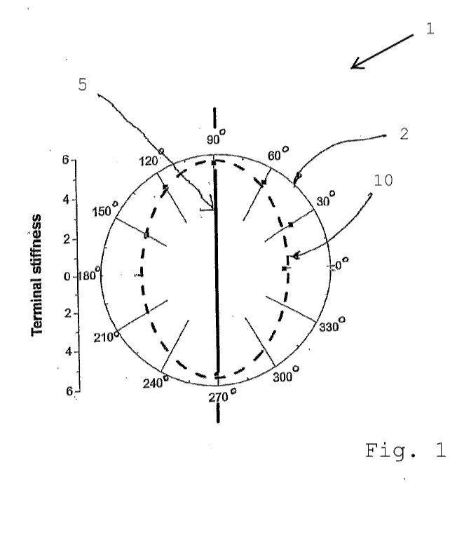

When the moduli of the high stiffness region of the stress-strain curves through a fixed point in various orientations are plotted in polar co-ordinates, the graph of mechanical properties with respect to testing direction is periodic. It is clear from FIG. 1 that these points join to form an ellipse shape 1.

These results substantiate the hypothesis that Langer's line 5 is the preferred orientation of the fibres within the reticular dermal tissue. The results as shown in FIG. 1 demonstrate that the direction of a local Langer's line 5 can be positively determined by multiple force-elongation tests. However, obtaining a complete set of load-extension curves in many directions is extremely time consuming.

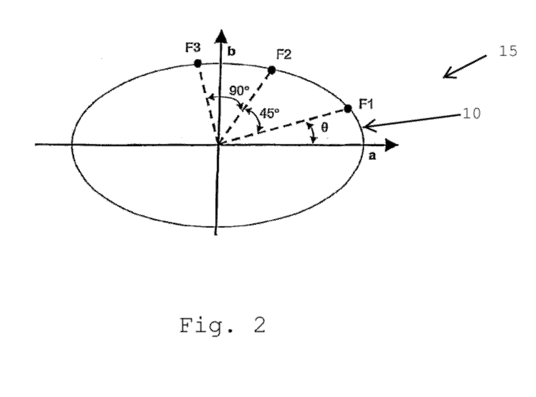

FIG. 2 shows the effect of limiting the number of such tests. In order to minimize the number of tests needed, a mathematical procedure may be adopted formulated using only 3 points F1, F2 & F3. It is hypothesized that the 3 data points will follow an ellipse 10. In order to find the equation of an ellipse that will best fit the 3 data points, all the calculations are performed in polar co-ordinates and the equation of the ellipse is given as follows:

$$\frac{{F}^{″} {cos}^{2} F sin 0}{{a}^{2} = 1} \\ where a = Major axis of the ellipse \\ \begin{matrix}b = Minor axis of the ellipse \\ = angle between any point on the ellipse and the major axis\end{matrix} \qquad ( 1 )$$

The first data point F1 at 0° is taken approximately along the direction of the skin's crease lines (which are known to be close to the Langer's line), and so this magnitude will be larger than F2 and F3. Therefore, it is expected that the major axis of ellipse to lie close to this point, and hence the value of 0 is expected to be small. By choosing a 45° sampling interval, one can ensure that the three data points will cover as much of one quadrant of the ellipse as possible for a high fitting accuracy. Alternatively, one may choose three lines at 60° angles, so that three data points will span at least two quadrants. Equation (2) can be obtained by substituting the test data to F1, F2, F3 and the angle into equation (1). Subsequently, a numerical solution can be found that will best satisfy equation (2).

$$\begin{matrix}F 12 a 2 sin e \\ \\ p 2 \text{,} {2}_{{c }^{′} in} {2}^{′} \\ ″ \\ \\ \\ 2 \\ \\ {F}^{2} {a}^{2} {sin}^{2} \\ \\ 3\end{matrix} \vert \begin{matrix} \\ \\ { }_{0} + Fi 2 b 2 {(}_{0.4 \_} Lr \\ \\ 4 ) \\ \\ 0 {+}^{\_ 71.} \\ \\ 2 \\ \\ \end{matrix} \vert \begin{matrix} \\ { }_{cos t} 0 , {\_}_{a} 2 b 2 \\ \\ + {F}^{2} {b}^{2} {cos}^{t} \\ \\ 2 \\ \\ + {F}^{2} {b}^{2} {cos}^{2} \\ \\ 3 \\ \end{matrix} \vert \begin{matrix} \\ \\ \\ \\ \\ \\ 0 {+}^{LI .} \\ \\ 4 \\ \\ \end{matrix} \vert \begin{matrix}= {a}^{2} {b}^{2} \\ \\ {a}^{2} {b}^{2} \\ \\ \\ \\ \\ \\ \\ \\ \end{matrix} \qquad ( 2 )$$

The fitting error is calculated by taking 0 to be accurate and finding the difference between the experimental data and the data on the ellipse at the same angle. The largest error among the three data points is taken as the fitting error.

Therefore, this ideal method of assessing the direction of the local Langer's line is to use the testing device to produce load-extension dataset at three different directions, at 45° or 60° each other. Then by using the mathematical principle indicated by equation (2), the polar equation of prospective ellipse is solved numerically. The direction of the Langer's line will correspond to the direction of the major axis of the ellipse.

Alternatively, the ellipse may be considered (relative to any convenient system of axes, such as any two orthogonal directions or the directions of two of the measurements) as represented by an equation of the form

ax²+2bxy+cy²=1. (3)

The extension due to unit force in the direction of a vector (X,Y) with X²+Y²=1 (that is, a unit vector) is then inversely proportional to ax^{e}+2bxy+cy², since a large radius of (3) in that direction corresponds to a small value of aX²+2bXY+cY². Given such extensions E₁, E2 and E3 in the respective directions of three vectors (X₁,Y₁), (X₂,Y₂) and (X₃,Y₃), we thus have

[symbol]

a linear problem in the three coefficients a, b and c. This has the solution

$$\begin{matrix}a - \\ 2 b \\ \end{matrix} \vert = \vert \begin{matrix}2 & XiY & & { }_{2} \\ X 2 & {X}_{2} {Y}_{2} & {Y}_{2}^{2} & \\ X \text{:} & {X}_{3} {Y}_{3} & {Y}_{3}^{2} & \end{matrix} \vert \vert \begin{matrix}1 \text{/} {E}_{1} \\ 1 \text{/} {E}_{2} \\ 1 \text{/} {E}_{3 \_}\end{matrix}$$

well defined if the three directions are distinct, and most robust if they are well separated. The Langer line through the current point is then the eigenline belonging to the smaller eigenvalue

$$A = a + c - V \overline{{( a - c )}_{2} + {0}^{2}} 2$$

that is, the line

(a 0.1.)x+by=0,

or equivalently

bx+(c−−0.1,)y=0.

Many alternative mathematical formulations will be recognized as equivalent to these by one skilled in the art.

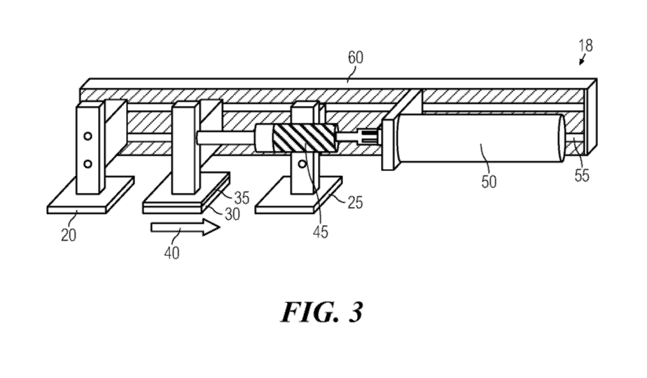

Therefore, in order to achieve the aforementioned results, a testing device 18 according to one embodiment of the invention is shown in FIG. 3. Three pads 20, 25 and 30 are attached to the skin of the patient. Two of the pads are fixed spatially to a bracket 60, with the third pad 30 in sliding engagement with said bracket 60. A servomotor 50 acts upon a worm gear 45 to apply a force to the slidable pad 30 to either bias it towards the distal pad 20 or the proximate pad 25. Recording of the applied force is measured through load cell 35, and in this embodiment electronically recorded (not shown).

Displacement may be measured through a displacement transducer. Thus a log of the application of force against displacement or time during the extension or compression 40 of the skin can be recorded. A preferred applied maximum strain of 50% may be adopted, to avoid patient discomfort, and also to ensure the integrity of the attachment means of the pads to the skin.

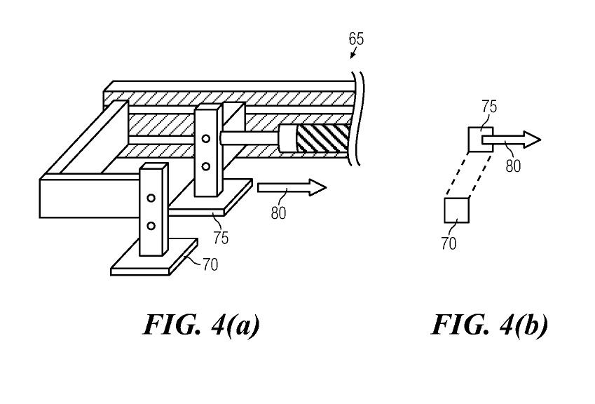

FIG. 4(a) shows an alternative arrangement of the testing device 65. Here the distal pad 70 is positioned at right angle to the application of force 80. Thus the slidable pad 75 will tend to stretch the skin to produce a shear effect, as shown in FIG. 4(b).

Whereas a plot of the results of the arrangement in FIG. 3 would provide a direct-characterisation of the relation between elongation and tension, the equivalent plot of force against positionally imposed strain for the arrangement of FIG. 4(a) would yield a characterisation of the relation between elongation and shear, again adding to the range of biomechanical properties offered by embodiments of the testing device of the present invention.

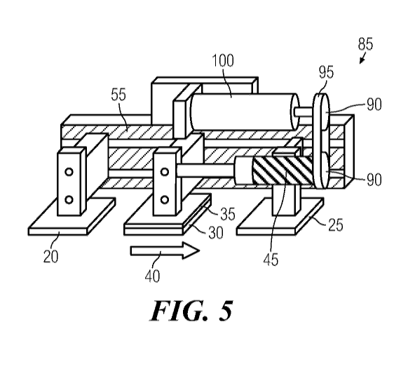

FIG. 5 shows an alternative arrangement 85 to the direct force application device of FIG. 3. Here, the servomotor 100 is placed above the gear 45, with the drive provided through a belt, or chain drive arrangement 90, 95. As with the arrangement of FIG. 3, the slidable pad is biased 40 towards the proximate pad 25, for direct force/elongation measurement.

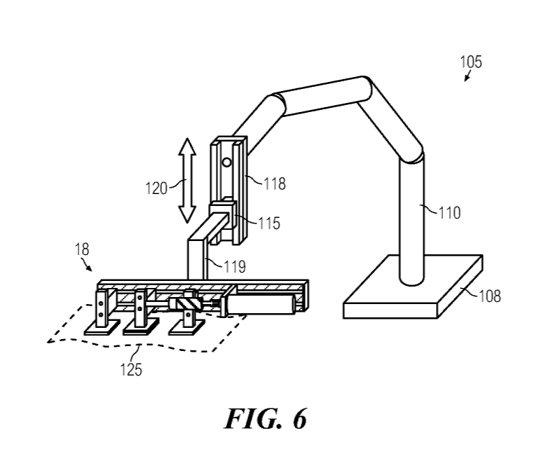

FIG. 6 shows an additional attachment to the overall assembly, whereby the testing device 18 is mounted to a positioning assembly 105. This positioning assembly 105 includes a bracket or platform 108 which may be attached to a stable external location, and a flexible articulated arm 110. At the distal end of the arm 110 is a holding arrangement 118, whereby the testing device 18 can be supported in a sliding 120 arrangement through slide 115. A further extension arm 119 is then used to offset the testing device 18 from the positioning assembly 105.

Thus, the positioning assembly 105 can position the testing device 18 in any number of arrangements without the human operator handling the device. The slide 115 enables the device 18 to rest horizontally on the skin 125 at its own weight, thereby standardizing the pressure that the pads 20, 25 and 30 presses onto the skin. This standardization and non-operator handling enable consistent and reproducible measurements to be taken.

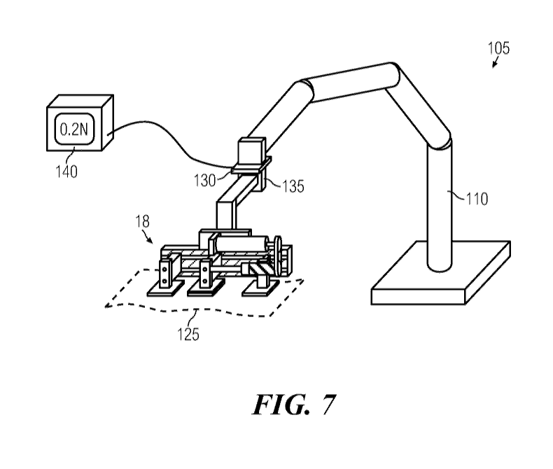

FIG. 7 shows a further arrangement of the positioning assembly 105, whereby the holding arrangement 118 of FIG. 6 is replaced with a holding engagement 135. The testing device 18 will preferably press onto the skin at a standard force during measurement. Otherwise, the readings may vary between samples. If the pressure is very high, then the skin beneath the pads will be overly compressed. This may cause the skin between the pads to push outward and affect the measurement. In addition, the load cell will also register an offset reading and contribute further to the error. Lastly, compressing the skin will cause the biological structures inside to press together and this will affect the mechanical behaviour. Conversely, if the pressure is very small such that the pad just lightly touches the skin, the skin attachment means may detach easily after a small strain. It follows that readings may be affected by the pressure on the skin, and different handling procedures of the operator. Therefore, standardization is very attractive for consistent and reproducible measurement results over time and between different operators.

In a further preferred embodiment, the load cell may also measure torque to make sure that all the pads press onto the skin at the same force; if there is any unevenness, a resultant torque will be registered. Alternatively, load cells placed beneath each pad may be used to detect a differential in pressure between the pads, and subsequently used to balance the pressures. The operator will press the device into the skin until a specified force and torque are registered at the load cell meter 140. Then measurement will start. This configuration enables the device to be placed at any angle to the surface.

In a further embodiment, different size pads may be used to minimize the “edge effect” during an in vivo experiment. It is suggested that increasing the “aspect ratio” (between the pad width and the distance between the pads) may reduce differences between in vivo and in vitro data. Thus, by selecting pads having a practically large aspect ratio, such as 2.5, the error contribution due to the surrounding materials in an in-vivo measurements environment may be minimized. Thus, attained results will be closer to the true characteristics of the materials, as measured in vitro (though some measurement such as shear response may become more difficult). This will permit comparison and normalization of data acquired with the present invention, against data acquired by the use of previously standard devices.

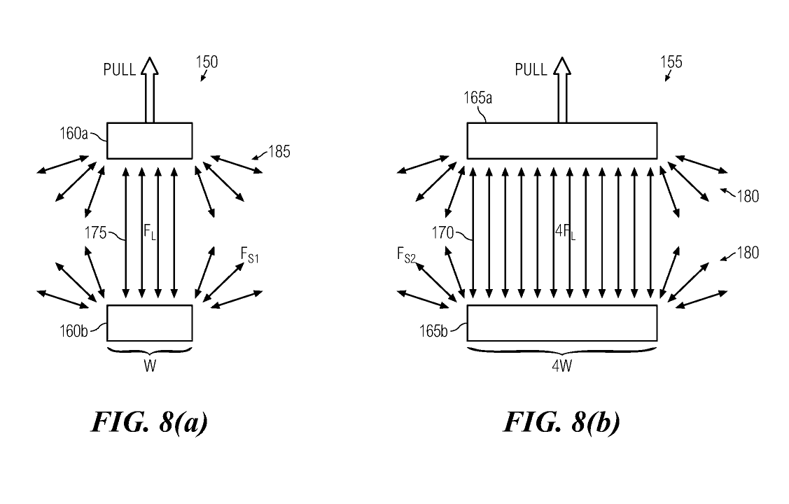

The following discussion makes reference to FIGS. 8(a) and (b). In an in-vitro measurement, the stress-strain property of a material can be accurately measured because the test sample is prepared to the appropriate size such that the grippers of the tensile tester cover the sample completely. Therefore, during pulling, the tension lines (principal directions of the stress tensor, for the larger eigenvalues) in the material are all aligned in the direction of applied force.

On the other hand, in an in-vivo measurement, as the pads (acting as grippers) move apart during measurement, the adjoining material is also deformed. Therefore, there will be additional tensor contributions from the adjoining material, and the measurement will not fully represent the stress-strain properties of the material between the pads.

The stress-strain data from an in-vivo test will have a higher magnitude compared to an in-vitro test. This is a problem for all in-vivo testers, such as extensometers. In one embodiment, the width of the pads may be large with respect to the separation between the pads. Increasing the aspect ratio (ratio of a pad's width to the pads' separation) may reduce the error between the stress-strain results obtained from in-vivo tests as compared to standard in-vitro tests.

With a large aspect ratio, during stretching, the tensor components 170 between the legs 165a,b are dominant compared to those contributing from the sides 180. The influence from the side tensors 180 becomes relatively minimal, and the measurement will be closer to the actual stress-strain between the pads. Therefore, the measured data will be closer to in-vitro data.

This can also be explained mathematically. Assume a situation where the width of the wide pads 165a,b (large aspect ratio configuration) is 4 times larger than the small pads 160a,b.

Let FL=Force contribution from linear tensors 175 between the small pads 160a,b Then the force from the principal tensor components 170 between the wide pads=4FL
Let Fs₁=Force contribution from the lateral tensor components 185 at the small pad due to stretching of the adjoining material
Let F32=Force contribution from the lateral tensor components 180 at the wide pad due to stretching of the adjoining material

### Therefore,

Stress at the small pads,

$$Small = \frac{\text{?}}{\text{?}}$$

$$\text{?}$$

$$\text{?} \text{indicates text missing or illegible when filed}$$

Stress at the wide pads, [symbol]
In an in-vitro test, Fs₁=0 or F=0, and so the stress t-viw

[symbol]

$$Small = \frac{+ F}{{ }_{si}} {F}_{si}$$

$$\text{?}$$

$$\text{?} \text{indicates text missing or illegible when filed}$$

[symbol]

In general, as the width of the wide pad increases, the error term will reduce and the result will gradually converge towards the in-vitro result. Therefore, the measurement will be more accurate.

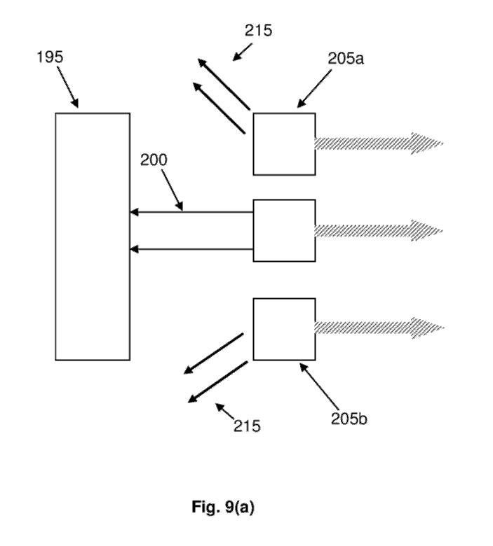

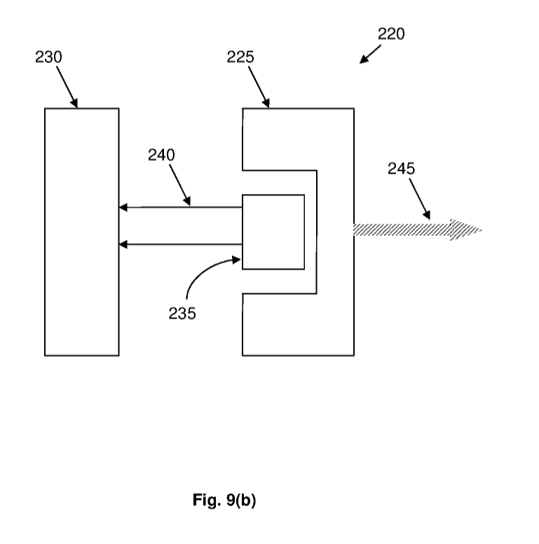

Alternative arrangements for the pads are shown in FIGS. 9(a) and 9(b). Here the concept of the “shield pad” is introduced. In the first embodiment, the pad arrangement 190 includes the stationary pad 195 according to the previous embodiments. Further included are peripheral pads 205a,b, which act as “shield pads to the sensor pad 200.

A typical extensometer has 2 pads (attached to the skin) that move apart during measurement. In this arrangement 190, forces measured in in-vivo are always higher than in-vitro ones for the same extension. In an in-vitro measurement, the material is excised and prepared such that the width is the same/smaller as that of the pads or grippers.

In in vivo measurements, the force measured is higher because the surrounding material is stretched together with the material between the pads. FIG. 9(a) shows simplified tensor lines 210, 215 to illustrate what goes on in an in-vivo measurement.
Since the desired data is the mechanical property of the skin 210 between the pads 210 and 195, the contributions due to 215 are undesirable. Furthermore, the “in-vitro” data is needed because:
1. Finite element modelling requires true material properties to simulate skin flap shrinkage.
2. In order to find true NL, elastic modulus and NT of skin
3. In-vitro data reflects the true uniaxial properties of the skin in the measured direction. If the measured data is influenced by the properties of skin in the other directions, then data interpretation is more difficult.

To the right of this arrangement, the upper peripheral pad 205a and lower peripheral pad 205b sandwich the sensor pad, which contains the load cell. These peripheral pads 205a,b effectively shield the sensor pad from the surrounding forces, and the load cell is mainly subjected to the forces 210 between pad 195 and pad 200. Therefore, the results measured will be much closer to the in-vitro result.

In an alternative embodiment of the “shield pad” concept, to further isolate the load cell from external forces, a C-pad 225 may be used for a complete shielding of the sensor pad 235, as shown in FIG. 9(b).

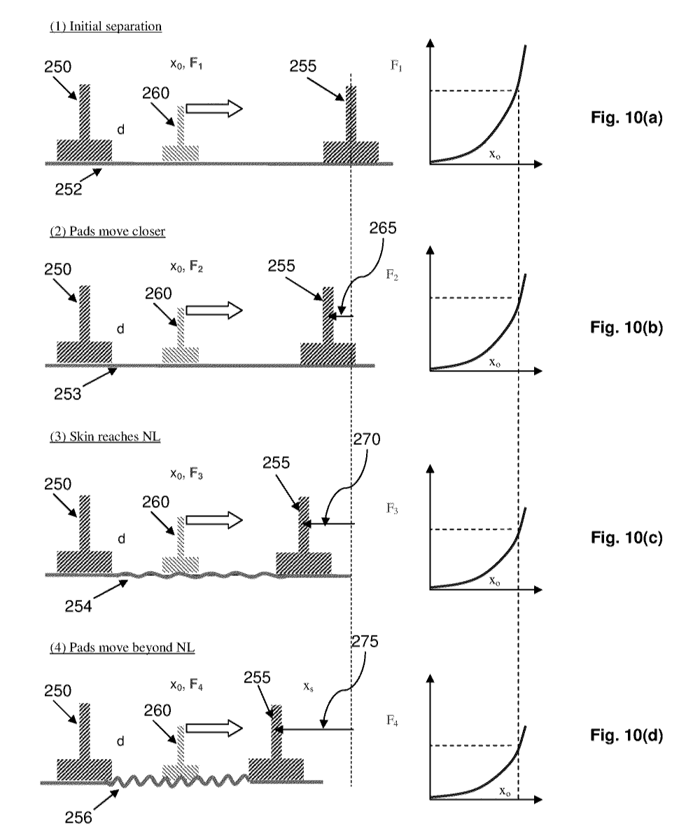

FIGS. 10 to 12 show a methodology to find the NL of skin in-vivo using the extensometer according to an aspect of the present invention.

In one embodiment of the methodology, FIGS. 10(a) to 10(d) shows a four stage process. Here, two large side pads 250, 255 are attached to the skin 252 while a load cell pad 260 measures the force at a specified extension (x₀) from a fixed distance (d) from the left pad. In this embodiment, for a distance between the pads 250, 255 of 60 mm, the fixed distance (d) may be in the range 10 to 30 mm, and the specified extension (x0) being in 10 mm. At stage 1, shown in FIG. 10(a), the force F₁ will be highest. As the side pads 250, 255 move together (denoted by xs) at stage 2, as shown in FIG. 10(b), the skin 253 in between will be slightly relaxed. Therefore, the force measured (F₂) at the same position (d) and same extension x₀ will be lower. It should be noted that the incremental movement of the pads (xs) may be about 1 mm.

When the pads 250, 255 move to stage 3, as shown in FIG. 10(c), the skin 254 in between reaches the natural length and will be completely relaxed. Hence, the force measured F3 will ideally reach the lowest value. At any subsequent distances (xs), the force measured will remain at the same value (F₄=F₃). On the other hand, if the skin 256 goes into compression, as shown in FIG. 10(d), after reaching the natural length, then the force measured will be higher (F4a>F3) 335.

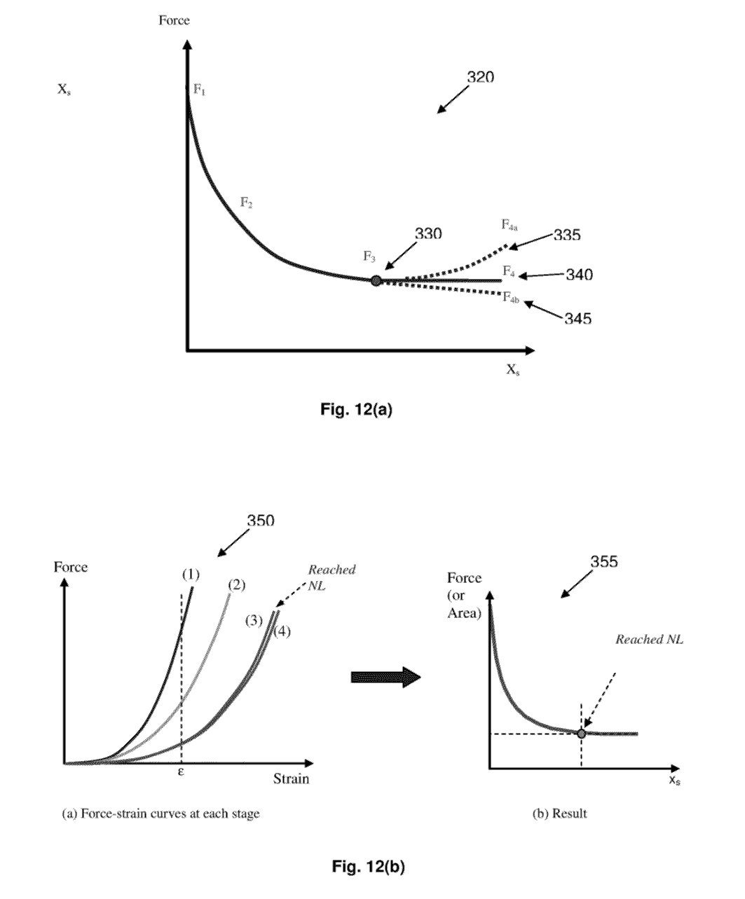

As shown in FIG. 12(a), in either of the cases above, a transition point 330 where the curve 310 goes flat 340 will be observed, with that transition point 330 corresponding to the natural length position. In certain circumstances, the curve may not become horizontal as expected, but the gradient may fall to a low value near zero, FM 345. The transition point may be taken as the point where the gradient falls to a specified threshold.

Following the methodology of FIG. 10(a) to (d), it may be necessary to remove the load cell pad 260 every time the side pads are moved together (xs). If the load cell pad 260 remains attached to the skin at distance (d) while the right pad is moved closer; the skin on both sides of the load cell pad may be unevenly distributed. In this case, the result may not be sufficiently accurate.

Further, the skin may wrinkle unevenly between the side pads 250, 255, with the skin nearer to the side pad 250 folding more than that near the middle.

This uneven wrinkling may create a problem for the force measurement at the load cell pad 260, unless it is always kept at the centre of the side pads 250, 255 so that the skin is evenly distributed on the left and right. However, since the load cell pad must be kept at a standard distance (d) from one side, the uneven wrinkling may cause the force measurement to be inaccurate.

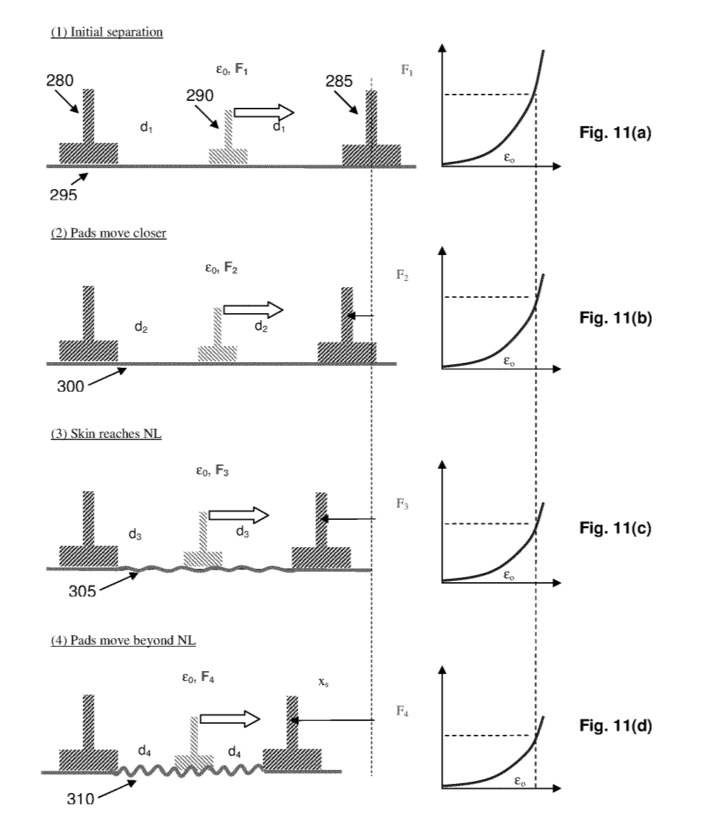

A solution is demonstrated in the further embodiment shown by the methodology of FIG. 11(a) to (d). Here the object is to think in terms of strain. This is done by keeping the load cell pad always at the centre, and to plot the result for force at the same strain (a), possibly in the range 5% to 100%, instead of force at the same extension. As shown in FIGS. 11(a) to (d), the distances d1 to d4 may be in the range 10 to 30 mm for a pad separation of 60 mm.

The expected result is illustrated in FIG. 12(b), where the force at a specified strain (a) for each curve is plotted against xₛ 350, where xₛ may be in 1 mm increments, as with the method shown in FIGS. 10(a) to (d). Instead of force, the energy (per unit length) of each curve at the specified strain may also be plotted 355. This energy is found by computing the area under the curves (up to the specified strain). In practice, the energy is a better parameter than force because this parameter is less subjected to measurement noise.

The problems caused by automation difficulty and uneven skin wrinkling may be solved in this alternative method, should the greater degree of accuracy be required. By keeping the load cell pad always at the centre, the distribution of skin to its left and right is always even. Therefore, the force measurement is accurate. Furthermore, there is no need to remove the load cell pad at every retraction of the side pads, thus making automation easy.

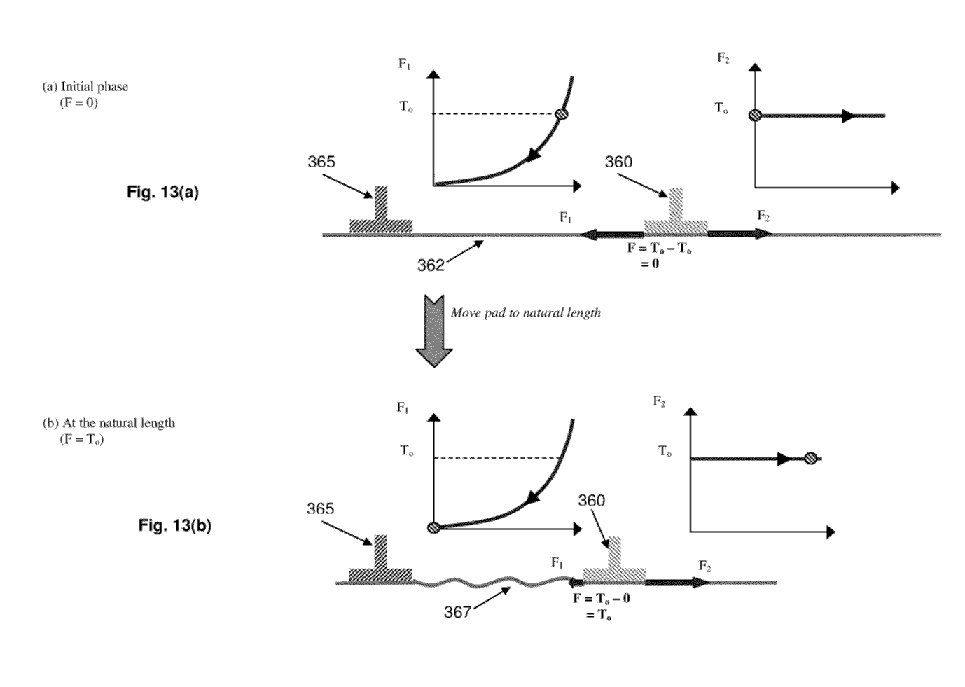

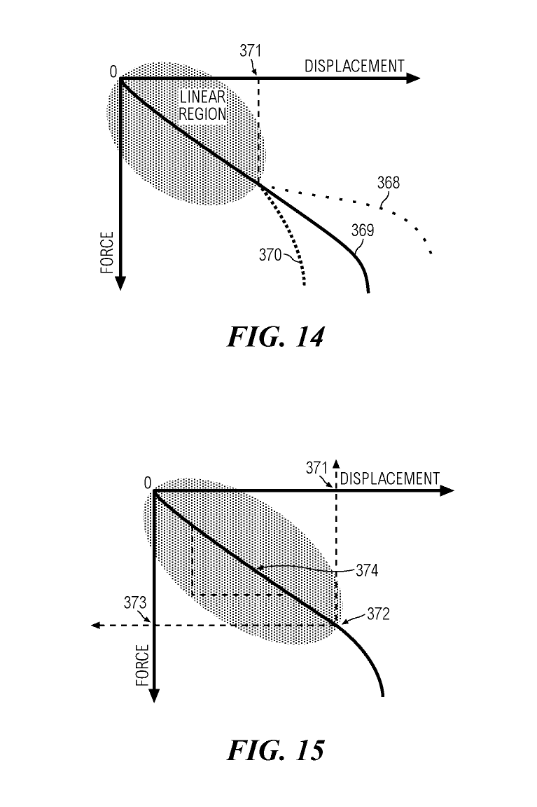

In a further embodiment, a method according to the present invention may be adopted to measure the NT, Elastic Modulus and NL of the skin using the “shield pad” embodiments, as shown in FIGS. 13(a) and (b). As mentioned earlier, the “shield pad” embodiments effectively reduce the force measured to one dimension.

The force measured by the extensometer is the difference between the skin tension on the left (F₁) and right (F₂) of the load cell 360, i.e. F2-F₁. When the extensometer is first attached to the skin 362, the load cell pad 360 reads no force since the natural tension (T₀) on the right cancels the natural tension on the left. A separation of the pads 360, 365 in the normalomstressed position may be approximately 25 mm.

As the load cell pad 360 is moved to the left towards the stationary pad 365, to compress the skin 367, the tension F₁ will gradually decrease in the typical J-profile. On the other hand, the tension F2 will remain approximately constant if the skin 367 is “infinitely” long on the right hand side. This is a reasonable assumption because the displacement applied is small compared to the much larger skin surface. If there are concerns that F2 may not remain constant during the compression, the C-pad shield 225, in particular, can be used to solve this problem.

When the load cell pad 360 reaches a position where the skin 367 in between the pads 360, 365 is at the natural length (NL). At this position, the tension F₁ is zero while F2 remains at the natural tension T₀. Therefore, the load cell will read the natural tension.

As the pad separation is further reduced, the skin in the middle undergoes compression. At this stage, three different cases may happen to the force-elongation reading (see FIG. 14). In the first case 368, the change in force becomes smaller with displacement, as the skin relaxes and folds gently upwards. In the second case 369, the change in force continues to increase linearly with displacement along the original curve. In the third case 370, the change in force becomes even greater with displacement, as the skin folds and squeezes together. Note that as more skin is being squeezed together, the force measured will eventually increase greatly and curve downwards because the skin tissue will squeeze tightly against each other.

In the first and second cases 368 and 370 above, the force-displacement curve changes direction from the initial straight line. In these cases, the transition point 371, which corresponds to the natural length, can be identified clearly. For the second case 369, the natural length will be overestimated, but it has been shown experimentally that this case is relatively rare.

When the natural length 371 is determined from above, the true origin 372 of the force-elongation behaviour of skin can be located (see FIG. 15). From here, the natural tension 373 can be deduced directly, while the gradient of the straight line 374 is the elastic modulus of the skin at the first phase.

1. An assembly for measuring in vivo biomechanical properties of skin, comprising a testing device, said testing device comprising:
a first pad array attachable to the skin;
a second pad attachable to the skin, at a known distance from the first pad array, said attachability of the pads to the skin to prevent relative movement between the respective pad and the skin to which it is attached;
a forcing means for applying a force to the first pad array, whilst said first pad array and second pads are attached to the skin, along a first axis connecting the first pad array and second pad, to induce a corresponding relative movement between a portion of the first pad array and the second pad due to deformation of the skin;
a force measurement device for measuring a force between the portion of the first pad array and the second pad as a result of the applied force; and
a displacement measurement device for measuring the corresponding induced movement.

2. The assembly according to claim 1, wherein the portion of the first pad array includes a sensor pad isolated from other pads forming the first pad array.

3. The assembly according to claim 2, wherein the first pad array further comprises at least two discreet pads placed peripheral to the sensor pad, said forcing means mounted to the discreet pads.

4. The assembly according to claim 2, wherein the first pad array further comprises a spreader pad of width greater than the sensor pad, said sensor pad placed adjacent to the spreader pad and intermediate the spreader pad and second pad, and said forcing means mounted to the spreader pad.

5. The assembly according to claim 4, wherein said spreader pad is C-shaped with the sensor pad located within a concave region of the C.
